(19) United States
(12) Patent Application Publication
(10) Pub. No.: US 2003/0044358 A1
(43) Pub. Date: Mar. 6, 2003

(54) Aerosol styling composition based on carboxyalkylalkylcellulose

(75) Inventors: Francoise Pataut, Paris (FR); Raquel DeCarvalho, Clichy (FR)

Correspondence Address: STEPTOE & JOHNSON LLP, 1330 Connecticut Avenue, N.W., Washington, DC, 20036, US

(73) Assignee: L'OREAL, Paris (FR)

(21) Appl. No.: 10/229,285

(22) Filed: Aug. 28, 2002

(30) Foreign Application Priority Data
Aug. 31, 2001 (FR) 0111343

(51) Int. Cl. A61K 7/06; A61K 7/11; A61K 9/00

(52) U.S. Cl. 424/47; 424/70.13

(57) ABSTRACT

The invention relates to styling compositions packaged in an aerosol device, comprising

a liquid phase containing, in dissolved form in a cosmetically acceptable liquid medium, at least one film-forming fixing polymer chosen from carboxyalkylalkylcelluloses, and

at least one propellant,

and also to a styling process using such a composition.

The present invention relates to a styling composition packaged in an aerosol device, containing, as fixing polymer, at least one carboxyalkylalkylcellulose, and also to a styling process using such a composition.

A very large number of fixing polymers, in particular anionic or nonionic polymers, that may be packaged, in dissolved form in a cosmetically acceptable medium, in an aerosol device are known in the field of styling.

When they have appreciable styling power, a large number of these polymers usually used nevertheless give the hair a rigid, stiff, unnatural appearance that is sought by fewer and fewer users.

This stiff appearance is partly due to the high glass transition temperature of the polymer, which deprives it of any plasticity at room temperature, but also due to the fact that, once the fixing polymer is dry, it sticks the hairs together, giving the hair the appearance of a “helmet”. The stiff appearance of the hairstyle only disappears after brushing or washing the hair.

Another problem arising in the formulation of fixing polymers in aerosol devices is linked to the viscosity of the liquid phases to be vaporized. Specifically, it is generally desirable to formulate the polymer in dissolved form in the liquid medium. However, the selection and use of good solvents for the fixing polymers usually involves a high viscosity of the liquid medium, which is reflected by a coarse spraying, a risk of blockage of the valves and a non-uniform distribution of the solution on the hair.

The Applicant has discovered that by using a particular family of known carboxylated cellulose ether derivatives as fixing polymers in styling compositions packaged in an aerosol device, the problems mentioned above may be overcome, i.e. solutions of fixing polymers that have relatively low viscosities, which are compatible with good spraying properties, may be prepared.

Moreover, the Applicant has found that when the carboxylated cellulose ether derivatives described in greater detail hereinbelow are used as fixing polymers, they give the hair satisfactory hold that is resistant to the usual movements of the head and the hair and to gusts of wind, but can readily be modelled by moderate mechanical stresses, for example by simply passing the hands through the hair.

This type of “brittle” fixing, due to inter-fibre linkages that are different from those created by usual fixing polymers, thus has the advantage of giving treated hair relatively supple and long-lasting hold without, however, giving the impression of rigidity usually observed for the usual fixing polymers.

One subject of the present invention is, consequently, a styling composition packaged in an aerosol device, comprising

a liquid phase containing, in dissolved form in a cosmetically acceptable liquid medium, at least one film-forming fixing polymer chosen from carboxyalkylalkylcelluloses, and

at least one propellant.

A subject of the present invention is also a styling process that involves spraying such a styling composition onto the hair and evaporating the cosmetically acceptable liquid medium.

The carboxyalkylalkylcelluloses used as fixing polymers in the styling compositions of the present invention are known per se. They are prepared, for example, by partial carboxyalkylation of cellulose followed by an alkylation of the carboxylated derivative obtained.

They preferably correspond to compounds with a repetition of units of formula (I)

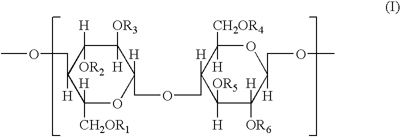

in which

R₁ to R₆ represent, independently of each other, a hydrogen atom, a C₁-C₄ alkyl group or a carboxy(C₁-C₄ alkyl) group, and

the average degree of polymerization is preferably between 30 and 300,

and the cosmetically acceptable salts of such a molecule.

As mentioned above, the carboxyalkylalkylcelluloses are preferably in dissolved form in the liquid phase of the styling compositions of the present invention. The solubility of the carboxyalkylalkylcelluloses, i.e. their ability to form macroscopically homogeneous transparent media in the presence of solvents, depends on their degrees of substitution, i.e. on the average number of carboxyalkyl groups and of alkyl groups per glucose unit, and also on the molecular mass of these polymers and the degree of neutralization of the carboxylic acid groups.

The carboxyalkylalkylcelluloses in non-neutralized, acid form are generally insoluble or difficult to dissolve in water, but show satisfactory solubility in polar organic solvents such as lower alcohols, for instance ethanol or isopropanol, or ethyl acetate, and in particular in water/alcohol mixtures.

The solubility in water of these cellulose derivatives may be readily increased by neutralizing the carboxylic acid functions with a cosmetically acceptable organic or mineral base chosen, for example, from alkali metal hydroxides, in particular sodium hydroxide or potassium hydroxide, and organic amines, in particular 2-aminomethylpropanol.

The carboxyalkylalkylcelluloses of the invention are preferably used in partially or totally neutralized form by addition of a base.

In one preferred embodiment of the present invention, the carboxyalkylalkylcellulose is a carboxymethylethylcellulose (CMEC), i.e. a carboxyalkylalkylcellulose of formula (I) in which at least one of the substituents R₁ to R₆ represents a hydrogen atom, at least one of the substituents R₁ to R₆ represents an ethyl group and at least one of the substituents represents a carboxymethyl group.

The carboxymethylethylcelluloses used in the styling compositions of the present invention preferably have a carboxymethyl group content of between 8% and 16% by weight, which corresponds on average approximately to one carboxymethyl group per two glucose units, and an ethyl group content of between 30% and 45% by weight, which is equivalent to a degree of substitution in the region of 3.

Examples of commercial products that may be mentioned include the carboxymethylethylcelluloses sold by the company Sanyo Chemical.

The cosmetically acceptable liquid medium into which the carboxyalkylalkylcelluloses are introduced includes, for example, water and cosmetically acceptable organic solvents that are preferably more volatile than water, such as lower alcohols, for example ethanol or isopropanol, acetone, methyl ethyl ketone, dichloromethane or ethyl acetate, and also mixtures thereof.

The cosmetically acceptable liquid medium is preferably an aqueous, alcoholic or aqueous-alcoholic medium, and in particular an aqueous-alcoholic medium containing at least 15% by weight of water, preferably from 15% to 90% by weight of water and in particular from 20% to 80% by weight of water.

The liquid phase, also known as the “fluid”, of the styling compositions of the present invention must have a minimum carboxyalkylalkylcellulose concentration allowing the deposition of a sufficient amount of fixing polymer.

In general, it has been found that a concentration of carboxyalkylalkylcellulose in the liquid phase that is in the range from 0.1% to 20% by weight, preferably from 0.2% to 10% by weight and even more preferably from 0.5% to 5%, is suitable for the invention.

The styling compositions of the present invention may be packaged in aerosol devices in the presence of any propellant usually used for preparing aerosol compositions. Propellants that are insoluble or partially soluble in the liquid phase, such as dimethyl ether, C₃-C₅ alkanes, 1,1-difluoroethane, mixtures of dimethyl ether and of C₃-C₅ alkanes, and mixtures of 1,1-difluoroethane and of dimethyl ether and/or of C₃-C₅ alkanes, will preferably be used.

The weight ratio of the liquid phase to the propellant in the aerosol compositions of the present invention is preferably between 20/80 and 80/20 and in particular between 30/70 and 70/30.

The styling compositions of the present invention may contain—in addition to one or more carboxyalkylalkylcelluloses—one or more other known anionic, nonionic, amphoteric or cationic film-forming fixing polymers, these fixing copolymers being in dissolved or dispersed (latex) form.

However, in one preferred embodiment of the styling compositions of the present invention, the carboxyalkylalkylcelluloses are the only fixing polymers present and are not combined with one or more other film-forming fixing polymers.

The styling compositions of the present invention may also contain one or more cosmetic or formulation additives usually used in cosmetics. Examples of such additives that may be mentioned include UV screening agents, fragrances, preserving agents, pigments and colorants, solubilizing agents, antifoams, vitamins, conditioners such as soluble silicones, which are dispersed or microdispersed, synthetic or natural, organic or mineral particles, or surfactants.

Needless to say, a person skilled in the art will take care to select this or these optional additional additive(s) and/or the amounts thereof such that the advantageous properties intrinsic to the invention are not adversely affected by the envisaged addition(s).

The styling compositions of the present invention are preferably hair lacquers.

The formulation example illustrates the present invention without, however, limiting it.

### EXAMPLE

### Aerosol Spray

2.5 g of carbomethylethylcellulose (Sanyo Chemical) are dissolved in a mixture of 43.6 g of water and 19 g of ethanol, the solution is neutralized with 0.44 g of 2-aminomethylpropanol and the fluid thus obtained is packaged with 35 g of dimethyl ether (propellant) in an aerosol device.

This lacquer, containing less than 55% by volume of volatile organic compounds, vaporizes readily and gives the hair satisfactory and long-lasting hold, which may be readily modelled by simply passing the hands through.

1. Styling composition packaged in an aerosol device, comprising
a liquid phase containing, in dissolved form in a cosmetically acceptable liquid medium, at least one film-forming fixing polymer chosen from carboxyalkylalkylcelluloses, and
at least one propellant.

2. Styling composition according to claim 1, characterized in that the carboxyalkylalkylcelluloses correspond to compounds with a repetition of units of formula (I) 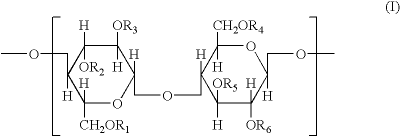
in which
R₁ to R₆ represent, independently of each other, a hydrogen atom, a C₁-C₄ alkyl group or a carboxy(C₁-C₄ alkyl) group,
and the cosmetically acceptable salts thereof.

3. Styling composition according to claim 1 or 2, characterized in that the average degree of polymerization is between 30 and 300.

4. Styling composition according to any one of the preceding claims, characterized in that the carboxyalkylalkylcellulose is a carboxymethylethylcellulose.

5. Styling composition according to claim 4, characterized in that the carboxymethylethylcellulose has a carboxymethyl group content of between 8% and 16% by weight and an ethyl group content of between 30% and 45% by weight.

6. Styling composition according to any one of the preceding claims, characterized in that the cosmetically acceptable medium is an aqueous, alcoholic or aqueous-alcoholic medium.

7. Styling composition according to claim 6, characterized in that the aqueous-alcoholic medium contains at least 15% by weight of water and preferably from 15% to 90% by weight of water.

8. Styling composition according to any one of the preceding claims, characterized in that the carboxyalkylalkylcellulose concentration in the liquid phase is between 0.1% and 20% by weight and preferably between 0.2% and 10% by weight.

9. Styling composition according to any one of the preceding claims, characterized in that it does not contain any film-forming fixing polymers other than the carboxyalkylalkylcelluloses of formula (I).

10. Styling composition according to any one of the preceding claims, characterized in that the propellant(s) is (are) chosen from dimethyl ether, C₃-C₅ alkanes, 1,1-difluoroethane, mixtures of dimethyl ether and of C₃-C₅ alkanes, and mixtures of 1,1-difluoroethane and of dimethyl ether and/or of C₃-C₅ alkanes.

11. Styling composition according to any one of the preceding claims, characterized in that the weight ratio of the liquid phase to the propellant is between 20/80 and 80/20 and preferably between 30/70 and 70/30.

12. Styling composition according to any one of the preceding claims, characterized in that it is a hair lacquer.

13. Styling process that involves spraying a styling composition, packaged in an aerosol device, according to any one of the preceding claims, onto the hair and evaporating the cosmetically acceptable liquid medium.
